(19) United States
(12) Patent Application Publication
(10) Pub. No.: US 2006/0162723 A1
(43) Pub. Date: Jul. 27, 2006

(54) Inhalation therapy device

(75) Inventors: Titus Selzer, Munchen (DE); Jurgen Pfrang, Kallmunz (DE); Markus Mornhinweg, Diessen (DE)

Correspondence Address: MERCHANT & GOULD PC, P.O. BOX 2903, MINNEAPOLIS, MN, 55402-0903, US

(73) Assignee: Pari GMBH Spezialisten Fur Effektive, Starnberg (DE)

(21) Appl. No.: 10/519,780

(22) PCT Filed: Jul. 3, 2003
(86) PCT No.: PCT/EP03/07114

§ 371 Date: Aug. 2, 2005

(30) Foreign Application Priority Data
Jul. 3, 2002 (DE) 102 29 889.0

(51) Int. Cl. A61M 11/00; A61M 15/00

(52) U.S. Cl. 128/200.14; 128/200.11; 128/203.12; 128/203.14

(57) ABSTRACT

The inventive inhalation therapy device (1) is designed in such a manner that the temperature of a liquid to be nebulized is measured and is taken into account in the form of nebuliztion schemata during the nebuliztion of the liquid.

The invention relates to an inhalation therapy device for nebulising liquids, in particular medicament-containing liquids, for applications in aerosol therapy.

The invention is based on the realisation that when nebulising medicament-containing liquids using jet nebulisers, the amount of nebulised liquid per unit of time, which is referred to as the “Total Output Rate (TOR)”, is dependent on the temperature of the liquid. Owing to nebulisation of the liquid, the temperature of the liquid to be nebulised decreases during a therapy session, and the TOR also decreases therewith. Other influences can also lead to a change in the temperature of the liquid. A constant TOR therefore cannot be assumed during the course of a therapy session, and it is thus difficult to determine the administered dose of medicament.

It is obvious to control the temperature of the liquid to be nebulised in a liquid storage container and to maintain said temperature at a predetermined value so that nebulisation of the liquid always occurs with defined boundary conditions and the TOR thus remains fixed over the duration of the therapy session.

It has already been suggested for other reasons as well to heat the liquid in an atomiser, such as, for example, in DE 30 43 537 A1. EP 0 582 124 A1 heads in a similar direction, in which the heating of incoming air to a jet nebuliser is described. The main focus of these solutions is, however, the provision of a heated aerosol, and no relation between temperature and TOR was described.

Temperature control of the medicament-containing liquid is complex since the desired control behaviour is only obtained both at the beginning of a therapy session, when there is a large amount of liquid, and towards the end of a therapy session, when there is a very small amount of liquid, if a corresponding amount of effort is made. Furthermore, in the case of some medicaments, rapid heating is undesirable since temperatures that are harmful to the effectiveness of the medicament can be locally obtained.

In view of the above, another way of exactly determining the amount of nebulised liquid is supposed to be revealed by the invention described herein, by means of which it is possible to reliably determine the dose of medicament administered during a therapy session.

To this end, the invention suggests detecting the temperature of the liquid to be nebulised and controlling nebulisation of the liquid in dependence on the detected temperature.

Control of nebulisation preferably occurs by means of a control device in such a manner that nebulisation only takes place at certain time intervals, the duration and/or frequency of which being set in dependence on the detected temperature by the control device. The relation between temperature and the duration/frequency of the nebulising intervals is stored in the control device, for example in the form of one or more maps in a memory means or in the form of a calculation method. These nebulisation schemata are empirically established and are adapted to the nebuliser and/or the medicament-containing liquid and/or the medicament and/or the therapy. Several nebulisation schemata can be stored in the control device and a user may select one of these.

The invention is particularly suitable for nebulisers in which generation of the aerosol can be activated or deactivated, i.e., for example, jet nebulisers and membrane nebulisers, without any significant start-up and shut-off processes occurring.

In a preferred embodiment, it is furthermore taken into consideration when controlling nebulisation that the concentration of the medicament in the stored liquid increases over the course of a therapy session. This effect is to be understood as follows in jet nebulisers: medicaments for nebulisers generally contain a mixture of different substances and a solvent which, in many cases, is water. The solvent is added to the air by means of jet nebulisation, i.e. the solvent vaporises into the air from the aerosol droplets. Large droplets flow back into the storage container, whereby an increase in the concentration of the medicament occurs depending on the duration of nebulisation and the temperature.

The invention is explained in more detail in the following by means of embodiments and referring to the figures.

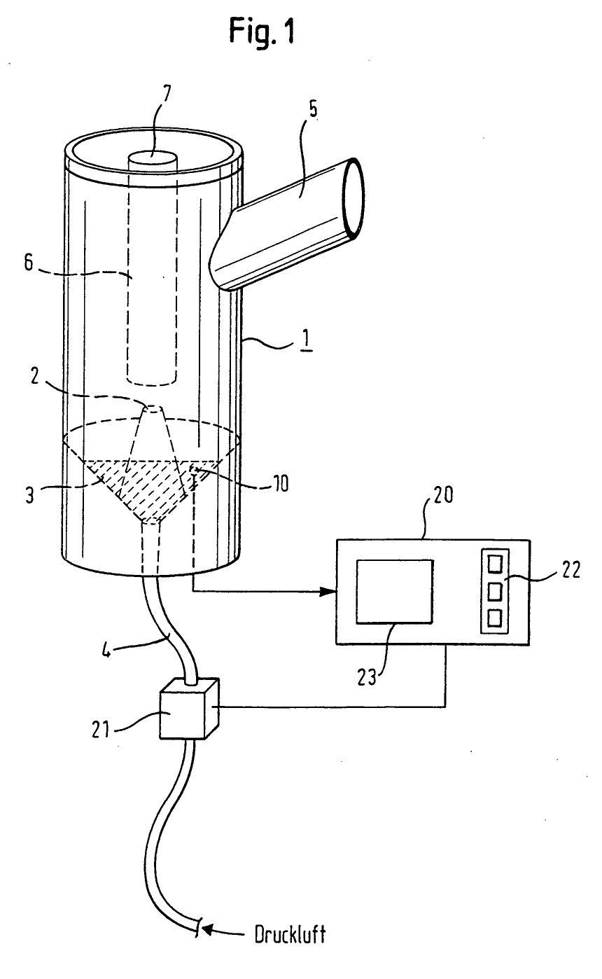

FIG. 1 shows an inhalation therapy device according to the invention having a jet nebuliser; and

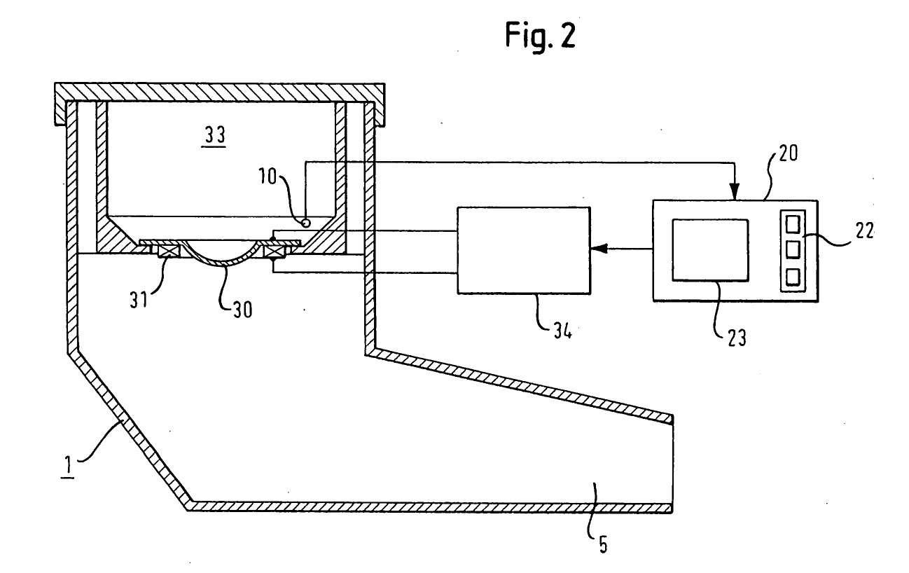

FIG. 2 shows an inhalation therapy device according to the invention having a membrane nebuliser.

FIG. 1 shows a nebuliser 1, namely a jet nebuliser having a supply air duct, as is known, for example, from EP 0 747 076 A2. The nebuliser 1 accommodates a nebulising nozzle 2 in its interior as the aerosol generator, which nebulises a liquid that is shown by means of hatching in FIG. 1 which is stored in a storage container 3. The nebulising nozzle 2 is supplied with compressed air for nebulisation via a tubing 4. This compressed air can be drawn, for example in a hospital, from a compressed air supply system or can be provided by means of a compressor (not shown). A patient inhales the aerosol generated by the nebulising nozzle 2 via a mouthpiece 5, whereby in the shown embodiment, ambient air flows into the interior of the nebuliser 1 via a supply air duct 6.

According to the invention, a sensor means 10 is provided, by means of which the temperature of the liquid is detected. In the embodiment shown in FIG. 1, the sensor means 10 is disposed directly in the liquid supply in the storage container 3. The output signal (measurement signal) of the sensor means 10 is supplied to a control device 20 which controls a valve means 21 disposed in the tubing 4, via which the compressed air is supplied to the nebulising nozzle 2. The valve means 21 influences the supply of compressed air to the nebulising nozzle 2, preferably in the form of a switch, in that it allows or prevents the supply of compressed air.

The control device 20 controls the valve means 21 in dependence on the temperature of the liquid detected by the sensor means 10 and effects an intermittent nebuliser operation which is dependent on the temperature. If the valve means 21 is open, compressed air flows to the nebulising nozzle 2 such that nebulisation of the liquid from the storage container 3 occurs. If the valve means 21 is closed, no compressed air reaches the nebulising nozzle 2, and thus nebulisation of the liquid from the storage container 3 does not occur either.

In a preferred embodiment, the control device 20 is designed such that the length of the time periods in which the valve means 21 is open owing to control by the control device 20 and in which nebulisation of the liquid occurs is, in each case, directly dependent upon the temperature of the liquid detected by the sensor means 10. In other words, each time interval in which nebulisation takes place is individually set by the control device 20, namely in dependence on the temperature of the liquid.

In a further preferred embodiment, the control device 20 is designed such that the length of each of at least two successive time periods in which nebulisation takes place is dependent on the temperature of the liquid detected by the sensor means 10.

The manner in which the control device 20 controls nebulisation is referred to as a nebulisation schema within the scope of this description. This is the set of rules which describes the relation between the detected temperature of the liquid to be nebulised and the manner of nebulisation, in particular the duration and frequency. In the simplest case, the set of rules merely comprises a map in which a time period is allocated to each temperature value or range. A memory means 23 is provided in the control device 20, in which the nebulisation schema or several nebulisation schemata are stored.

In a further preferred embodiment, the control device 20 comprises a selecting means 22, for example a group of switches, by means of which the patient selects a medicament to be nebulised. The control device 20 then comprises several nebulisation schemata in the memory means 23, which are stored there for the selectable medicaments. Different nebulisation schemata for one medicament can also be stored in the memory 23, of which one can be selected by a user by means of a correspondingly designed selecting means 22.

In a preferred embodiment, it is furthermore taken into consideration when controlling nebulisation that during a therapy session, the concentration of the medicament in the stored liquid increases. The control device 20 is designed therefor in such a manner that the duration of the therapy session is established and is consulted together with the measured temperature for control of nebulisation. Concentration courses over the duration of the therapy can be empirically determined in dependence on the duration and temperature and can serve as a basis for setting the duration/frequency of the nebulisation intervals by the control device 20. These relations are also preferably stored in the memory means 23 of the control device 20.

The basis for a further increase in dosage accuracy is created in this manner. The reason for this is that the longer a therapy session lasts, the higher—depending on the temperature—the concentration of the medicament in the still stored liquid is. Control of nebulisation then occurs, for example, such that shorter nebulisation intervals or fewer nebulisation intervals occur so that the dose remains the same. Alternatively, control of the nebulisation intervals can occur as described above, however taking into consideration the increased concentration when calculating the dose of the medicament.

The initial concentration of the medicament in the liquid to be nebulised can be fixedly specified, however the inhalation therapy device according to the invention is set to a specific medicament at a specific concentration. Alternatively, it is possible to create an input/selection possibility, in which, for example, the selecting means 22 is correspondingly designed in such a manner that a user can select the medicament and the initial concentration.

In an alternative embodiment which is shown in FIG. 2, the nebuliser 1 comprises a nebuliser membrane 30 as the aerosol generator, which is caused to oscillate by an oscillation generating device 31, for example a piezo ring element, whereby a liquid disposed on one side of the membrane, which is stored in a storage container 33, is nebulised through the membrane 30. The oscillation generating device 31 is excited by an excitation device 24 which is connected for this purpose with the piezo ring 34. Membrane nebulisers of this type are known, for example, from DE 199 53 317 or EP 0 615 470.

As is the case for the first embodiment, a sensor means 10 is provided according to the invention, which detects the temperature of the liquid stored in the storage container 33 and supplies a measurement signal to the control device 20. The control device 20 controls the excitation device 34 in dependence on the detected temperature of the liquid such that the piezo ring element causes the membrane to oscillate in accordance with the control by the control device 20. The liquid disposed on the membrane is nebulised in this manner in dependence on the control by the control device 20 and thus in dependence on the temperature detected by the sensor means 10.

The manner of control occurs according to the same criteria as explained in connection with the first embodiment, however adjusted to the membrane nebuliser.

1. An inhalation therapy device comprising
a. a nebulising device for nebulising a liquid stored in a storage container;
b. a sensor means for detecting the temperature (T) of the liquid; and
c. a control device for controlling nebulisation of the liquid by the nebulising device in dependence on the temperature (T) detected by the sensor means.

2. An inhalation therapy device according to claim 1, wherein said nebulising device comprises a nebulising nozzle to which compressed air can be supplied to nebulise the liquid, and that said control device comprises a valve means which influences, in particular allows or interrupts, the supply of compressed air to the nebulising nozzle.

3. An inhalation therapy device according to claim 1, wherein said nebulising device comprises a nebuliser membrane which can be caused to oscillate by means of an oscillation generating device so as to nebulise a liquid disposed on one side of the membrane, that said control device is connected with an excitation device which excites said oscillation generating device, and that said control device controls, in particular activates and deactivates, said excitation device.

4. An inhalation therapy device according to claim 1, wherein said control device comprises a memory means, in which information regarding the relation between the detected temperature and the manner of control of the nebulising device (nebulisation schema), in particular as regards the duration and/or frequency of nebulisation, is stored.

5. An inhalation therapy device according to claim 4, wherein a plurality of nebulisation schemata are stored in the memory means.

6. An inhalation therapy device according to claim 5, wherein the plurality of nebulisation schemata can be used for different nebulisers and/or medicaments and/or therapies.

7. An inhalation therapy device according to claim 1, wherein the control device comprises a selecting means, via which one of the nebulisation schemata can be selected by a user.

8. An inhalation therapy device according to claim 1, wherein the control device controls the nebulising device such that nebulisation takes place in time intervals whose length is dependent on the temperature of the liquid to be nebulised.

9. An inhalation therapy device according to claim 1, wherein said control device controls the nebulising device such that nebulisation takes place in time intervals whose frequency is dependent on the temperature of the liquid to be nebulised.

10. An inhalation therapy device according to claim 1, wherein when controlling the nebulising device, the control device takes into consideration an increase in the concentration of the medicament in the stored liquid occurring during a therapy session.

11. An inhalation therapy device according to claim 10, wherein said control device comprises a memory means in which information regarding the control taking into consideration the temperature-dependent increase in the concentration of the medicament (nebulisation schema) is stored.

12. An inhalation therapy device according to claim 10, wherein said control device takes into consideration the temperature-dependent increase in the concentration of the medicament by shortening the nebulisation intervals.

13. An inhalation therapy device according to claim 10, wherein the control device takes into consideration the temperature-dependent increase in the concentration of the medicament by reducing the frequency of the nebulisation intervals.

14. An inhalation therapy device according to claim 10, wherein the selecting means of the control device is designed for selecting a medicament and/or an initial concentration.

15. An inhalation therapy device according to wherein the control device determines the administered dose of the medicament based on the duration/frequency of the nebulisation intervals and the concentration of the medicament in the stored liquid, and ends nebulisation upon reaching a predetermined dose.
